(19) United States
(12) Patent Application Publication
(10) Pub. No.: US 2026/0231992 A1
(43) Pub. Date: Aug. 13, 2026

(54) A PROCESS FOR MYCOTOXIN DETOXIFICATION USING LACTOCOCCUS LACTIS

(71) Applicant: COUNCIL OF SCIENTIFIC & INDUSTRIAL RESEARCH, New Delhi (IN)

(72) Inventors: Vishwambar Digambar NAVALE, Pune (IN); Rao VAMKUDOTH, Pune (IN); Balasaheb Raghunath BORADE, Pune (IN); Ravindar KONTHAM, Pune (IN)

(73) Assignee: COUNCIL OF SCIENTIFIC & INDUSTRIAL RESEARCH, New Delhi (IN)

(21) Appl. No.: 19/157,609

(22) PCT Filed: Feb. 19, 2024
(86) PCT No.: PCT/IN2024/050175

§ 371 Date: Aug. 18, 2025

(30) Foreign Application Priority Data
Feb. 20, 2023 (IN) 202311011678

(51) Int. Cl. A23L 5/20; A23B 2/783; C12N 1/205; C12R 1/46

(52) U.S. Cl. CPC A23L 5/28; A23B 2/783; C12N 1/205; C12R 2001/46

(57) ABSTRACT

The present invention relates to a method of in-vitro detoxification of multiple mycotoxins using Lactococcus lactis subsp. lactis which is isolated from traditional milk samples. The mycotoxins as disclosed in the present invention are ochratoxin A (OTA), zearalenone (ZEA), fumonisins B1 (FB1), deoxynivalenol (DON) and citrinin (CIT). In one aspect present invention relates to process of inactivation of spore germination and inhibition of biosynthesis of fumonisin B1 micotoxin using Lactococcus lactis subsp. lactis. The species Lactococcus lactis subsp. lactis produces novel antimicrobial metabolites such as 2,4-diotert-butylphenol, Pyrazinamide, and 3-5-dihydroxy-6-methyl-2,3-dihydro-4H-pyran-4-one.

### FIELD OF THE INVENTION

The present invention relates to a process for the detoxification of multiple mycotoxins. More particularly, the present invention relates to a process of in-vitro detoxification of multiple mycotoxins by using Lactococcus lactis subsp. lactis to ensure the food safety and human health.

### BACKGROUND OF THE INVENTION

Fungi are a group of eukaryotic organisms that include the genera Aspergillus, Fusarium, Penicillium, and Alternaria that are the major mycotoxin producers in various agricultural products, food matrices, and oil seeds. Aflatoxins (AFTs), ochratoxin A (OTA), zearalenone (ZEA), fumonisins (FBs), trichothecenes (TRIs; deoxynivalenol (DON) and nivalenol (NIV), citrinin (CIT), patulin (PAT) are major contaminants of food and feed grains. Mycotoxins contamination is one of the serious global issues that lead to huge economic losses. A wide range of food grains are contaminated with more than one mycotoxin during both pre- and post-harvest period. Mycotoxins chemotypes, FB1, ZEA, OTA, DON, and CIT are found in cereals and other food products like beans, coffee, wine, beer, dairy products, etc., whereas FB1, DON, and ZEA are frequently found in meat, chicken, and eggs. The exposure of humans and animals to mycotoxin-defiled food could result in acute and chronic effects such as teratogenicity, estrogenic effects, carcinogenesis, immune suppression, and neurotoxicity. Mycotoxins are low molecular weight secondary metabolites that are thermostable and cannot be removed during normal food processing (80-120° C.). European commission, food and agricultural organization, US Food and Drug Administration (FDA), and Food Safety and Standards Authority of India (FSSAI) set up regulations of mycotoxins in food and feed. Recent reports suggests that, about 88% of cereal samples and pulses are contaminated with one or more mycotoxins. Co-occurrence of mycotoxins in food and feed causes adversary and synergetic impacts that negatively affect animal digestion, fertility, and death rate, and also enhance the toxicity which naturally occurs with AFTs and FUM; DON and ZEA, and FUM+ZEA. There are various mycotoxin management strategies available such as physical, chemical, and biological in combating mycotoxins load in the food and feed system. However, mycotoxins management by physical and chemical measures may affect the nutrient quality and the consumer's health and also increase the production cost. Whereas, biological approaches are safer and more eco-friendly than physical and chemical approaches. However, probiotics, or lactic acid bacteria (LAB) are generally recognized as safe (GRAS) and have potential antifungal and mycotoxin detoxification properties in food and feed and many health benefits to the consumers and can be used as a food-grade preservative without compromising the quality and biochemical properties of the food.

The various prior art documents state that several genera of bacteria Lactobacillus, Lactococcus, Bifidobacterium, Enterococcus, and Pediococcus showed probiotic characteristics and had antifungal and mycotoxin detoxifying properties. L. plantarum inhibits the growth of F. verticillioides and FUM biosynthesis (73 to 99%) (De Melo et al. 2019). L. lactis results in 88% reduction of ZEA mycotoxin (Krol et al. 2018), and another strain of L. lactis resulting in the lowering of DON (23-40%), FB2 (100%) and there is no detoxification of FB1 (Niderkorn et al. 2006). Similarly, Niderkorn et al. (2009) reported that L. lactis subsp. cremoris MG1363 strain leads to complete removal of FB1.

Thus, in consideration of mycotoxin contamination and their risk to health/environment, neutralization of multiple mycotoxins in food and feed is important and hence it is necessary to find out a solution for detoxification of multiple mycotoxins which doesn't affect the nutrient quality and the consumer's health and are also economical.

### OBJECTIVES OF THE INVENTION

The main objective of the present invention is to provide a method for in-vitro detoxification of multiple mycotoxins.

Another object of the present invention is to provide a method of in-vitro detoxification of multiple mycotoxins using Lactococcus lactis subsp. lactis.

Another object of the present invention is to provide a process for in-vitro mycotoxin detoxification of various mycotoxins such as FB1, ZEA, CIT, DON, and OTA or combinations thereof.

Yet another object of the present invention is to provide a process for in-vitro mycotoxin detoxification of various mycotoxins such as FB1, ZEA, CIT, DON, and OTA or combinations thereof by using live cell pellet and fermented cell-free supernatant (CFS) of Lactococcus lactis subsp. lactis which contains new compounds selected from 3,5-dihydroxy-6-methyl-2,3-dihydro-4H-pyran-4-one, pyrazine-2-carboxamide and 2,4-di-tert-butylphenol, or combinations there of from Lactococcus lactis subsp. lactis.

### SUMMARY OF THE INVENTION

This summary is provided to introduce a selection of concepts in a simplified form that are further described below in Detailed Description section. This summary is not intended to identify key features or essential features of the subject matter, nor is it intended to be used as an aid in determining the scope of the subject matter.

In a general aspect, the present invention provides a method of in-vitro detoxification of multiple mycotoxins using Lactococcus lactis subsp. lactis (GenBank accession number MG917752), which is isolated from traditional milk samples (location: Kolhapur, Maharashtra, India). The mycotoxins as disclosed in the present invention are mycotoxins such as OTA, ZEA, and FB1, DON, and CIT.

The mycotoxins that contaminate a wide range of food grains and causes acute and chronic effects on humans, and livestock, and hence it is necessary to detoxify the food grains to control their adverse effects on consumers.

In one aspect the present invention relates to in vitro detoxification of multiple mycotoxins using single species and a single treatment.

In one aspect the present invention relates to a composition for in-vitro detoxification of mycotoxins, said composition comprises live cell pellets (CP) of Lactococcus lactis subsp. lactis; and/or fermented cell-free supernatant (CFS) comprising one or more metabolites produced by Lactococcus lactis subsp. Lactis in a solvent.

In an aspect of the present invention, the composition comprises the CP of Lactococcus lactis subsp. lactis is in a concentration in the range of 5-15% w/v; and the fermented cell-free supernatant (CFS) is in a concentration in the range of 5-15% v/v.

In an aspect of the present invention, the one or more metabolites produced by Lactococcus lactis subsp. lactis are selected from the group consisting of 3,5-dihydroxy-6-methyl-2,3-dihydro-4H-pyran-4-one, pyrazine-2-carboxamide and 2,4-di-tert-butylphenol.

In one more aspect, the present invention provides Lactococcus lactis subsp. lactis strain which produces novel metabolites such as 2,4-di-tert-butylphenol (1), 3,5-dihydroxy-6-methyl-2,3-dihydro-4H-pyran-4-one (2), Pyrazine-2-carboxamide (3).

In another aspect, the 2,4-di-tert-butylphenol (1), 3,5-dihydroxy-6-methyl-2,3-dihydro-4H-pyran-4-one (2), Pyrazine-2-carboxamide (3) produced from Lactococcus lactis subsp. lactis strains are present in CSF and hence are responsible for the detoxification of multiple mycotoxins.

In another aspect, the present invention relates to the inhibition of spore germination in an in-vitro condition using CP and CFS of the Lactococcus lactis subsp. lactis.

In another aspect, the present invention relates the use of L. lactis subsp. lactis as a food and feed additive for the betterment of food safety.

In another aspect, the present invention relates to a food additive composition for inhibition of microbial spore germination in a food product, said food additive comprises live cell pellets (CP) of Lactococcus lactis subsp. lactis; and/or fermented cell-free supernatant (CFS) comprising one or more metabolites produced by Lactococcus lactis subsp. Lactis in a solvent, wherein the food additive is a preservative.

In another aspect, the present invention relates to a process for in vitro detoxification of a food sample, said process comprising: treating the composition as disclosed herein with the food sample containing mycotoxins by spraying the composition onto said sample followed by incubating at a temperature in the range of 35 to 55° C. for a time period in the range of 0.5 to 24 hrs to obtain detoxificated sample.

In yet another aspect, the present invention relates to the inhibition of the mycotoxins biosynthesis pathway in an in-vitro condition using CP and CFS of Lactococcus lactis subsp. lactis.

In yet another aspect, the present invention relates to process for the production of the composition for in-vitro detoxification of mycotoxins, said process comprising the steps of:
a. isolating Lactococcus lactis subsp. lactis (MG917752) from raw milk by using serial dilution;
b. fermenting, Lactococcus lactis subsp. lactis (MG917752) of step b) at a temperature of 32-37° C. for 24 to 48 hours in a growth medium;
c. incubating, Lactococcus lactis subsp. Lactis (MG917752) followed by fermentation to get grown biomass crude mixture of Lactococcus lactis subsp. lactis based live cell pellets, and fermented cell-free supernatant (CFS) comprising one or more metabolites produced by Lactococcus lactis subsp. Lactis; and
d. filtrating fermented broth to separate live cell pellet (CP) containing Lactococcus lactis subsp. lactis (MG917752) and cell-free supernatant (CFS) of Lactococcus lactis subsp. lactis (MG917752); and
e. preparing the composition by physical mixing of said live cell pellets (CP) containing Lactococcus lactis subsp. lactis (MG917752) and/or said cell-free supernatant (CFS) of Lactococcus lactis subsp. lactis (MG917752) in a solvent to obtain the composition.

In yet another aspect, the present invention relates to the isolation of Lactococcus lactis subsp. lactis (MG917752) from raw milk comprises the steps of:
a. Providing raw milk;
b. Homogenising the raw milk with sterile distilled water containing 0.85% NaCl and 0.1% peptone;
c. Preparing serially diluted samples from the homogenized raw milk and spreading the diluted samples on Man, Ragosa, and Sharpe (MRS) agar plate;
d. Incubating the MRS agar plates at 32° C. to 37° C. for 24 to 48 h under aerobic conditions;
e. Picking up a typical Lactococcus characteristics colonies and stricked on MRS agar plates followed by Gram's staining and microscopic observation;
f. Confirming the strains of L. lactis subsp. lactis (MG917752) by molecular identification using 16s RNA gene.

### BRIEF DESCRIPTION OF THE DRAWINGS

FIG. 1 depicts ¹H NMR Spectrum (400 MHz, CDCl₃) of bioactive compound 1.

FIG. 2 depicts ¹³C NMR Spectrum (101 MHz, CDCl₃) of bioactive compound 1.

FIG. 3 depicts HRMS of bioactive compound 1.

FIG. 4 ORTEP diagram of bioactive compound 1.

FIG. 5 depicts ¹H NMR Spectrum (400 MHz, CDCl₃) of bioactive compound 2.

FIG. 6 depicts ¹³C NMR Spectrum (101 MHz, CDCl₃) of bioactive compound 2.

FIG. 7 ORTEP diagram of bioactive compound 2a.

FIG. 8 depicts ¹H NMR Spectrum (400 MHz, CDCl₃) of bioactive compound 3.

FIG. 9 depicts ¹³C NMR Spectrum (101 MHz, CDCl₃) of bioactive compound 3.

FIG. 10 shows ORTEP diagram of bioactive compound 3.

FIG. 11 depicts Effect of cell free supernatant of Lactococcus lactis subsp. lactis on growth of Fusarium verticillioides.

FIG. 12 depicts Effect of cell free supernatant of Lactococcus lactis subsp. lactis on FUM1 gene expression in Fusarium verticillioides.

FIG. 13 shows molecular identification of L. lactis subsp. lactis by 16s rRNA by Polymerase chain reaction (PCR).

FIG. 14 represents structure elucidation of new bioactive metabolites.

### DETAILED DESCRIPTION OF THE INVENTION

The deposition of microbe “Lactococcus lactis subsp. lactis (MG917752)” is done at authority National Centre for Cell Science (NCCS), Pune, MH, India on 9 Feb. 2023, and the details are provided below:

| | Processing | | |
|---|---|---|---|
| S. | Reference | Strain | Accession |
| No. | Number | designation | number |
| 1 | D_FEB_23_080 | BIONCL17752 | MCC 0263 |

** The obtained sequences were blasted at NCBI in Basic Local Alignment Search Tool (BLAST), and were showed 100% similarity with Lactococcus lactis subsp. lactis. The obtained sequences were deposited in NCBI with GenBank accession number MG917752.

In main embodiment, the present invention relates to a method of in-vitro detoxification of various mycotoxins using Lactococcus species. Wherein, the Lactococcus lactis subsp. lactis which is isolated from traditional milk samples (location: Kolhapur, Maharashtra, India). The mycotoxins as disclosed in the present invention are mycotoxins such as ochratoxin A (OTA), zearalenone (ZEA), fumonisins B1 (FB1), deoxynivalenol (DON) and citrinin (CIT).

In one aspect present invention relates to in-vitro detoxification of multiple mycotoxins using single species.

In one embodiment, the present invention relates to the process of detoxification of multiple mycotoxins comprising the steps of:
1. isolating, Lactococcus lactis subsp. lactis (MG917752) from raw milk,
2. fermenting, Lactococcus lactis subsp. lactis for 24 to 48 hours at 32 to 37° C. in de Man Rogosa Sharpe medium (MRS),
3. incubating fermented broth containing L. lactis subsp. Lactis,
4. filtrating fermented broth to separate cell pellet (CP) and cell-free supernatant (CFS), and
5. employing said CP and CFS against multiple carcinogenic mycotoxins FB1, ZEA, OTA, DON, and CIT.

In preferred embodiment, the present invention relates to the process of detoxification of multiple mycotoxins comprising the steps of:
1. isolating, Lactococcus lactis subsp. lactis (MG917752) from raw milk by using serial dilution comprising steps of:
   a. homogenizing, 1 ml of milk with 9 ml of sterile distilled water containing 0.85% NaCl and 0.1% peptone;
   b. preparing, 10-fold serial dilution from 10⁻¹ to 10⁻⁶ and spreading a volume of 0.1 ml of appropriate dilutions on Man, Ragosa, and Sharpe (MRS) agar plate;
   c. incubating, the above plates at 32° C. to 37° C. for 24 to 48 h under aerobic conditions;
   d. picking up a typical Lactococcus characteristics colonies and stricked on MRS agar plates followed by Gram's staining and microscopic observation; and
   e. confirming, strain of L. lactis subsp. lactis by molecular identification using 16S r-RNA gene,
2. fermenting, L. lactis subsp. lactis of step 1 at a temperature of 32-37° C. for 24 to 48 hours;
3. incubating, L. lactis subsp. lactis till 24-48 hours to get good growth and biomass;
4. filtrating, fermented broth to separate cell pellet (CP) and cell-free supernatant (CFS); and
5. employing said CP and CFS against multiple carcinogenic mycotoxins.

In yet another preferred embodiment, the present invention discloses the use of CP and CFS obtained from fermented MRS medium containing Lactococcus lactis subsp. against the detoxification of multiple mycotoxins. The Lactococcus lactis subsp. lactis of the present invention is first isolated from raw milk and then is allowed to be fermented in an MRS medium.

In another embodiment, the present invention discloses the use of fermented CS and CSF of Lactococcus lactis subsp. lactis against the invitro detoxification of various mycotoxins. In yet another embodiment, the present invention relates to the inhibition of spore germination in an in-vitro condition using CP and CFS of Lactococcus lactis subsp. lactis of the present invention.

In yet another embodiment, the present invention relates to the inhibition of mycotoxin FB1 biosynthesis pathway in an in-vitro condition using CP and CFS of Lactococcus lactis subsp. lactis as disclosed in the present invention.

In an embodiments, the present disclosure provides a composition for in-vitro detoxification of multiple mycotoxins, said composition comprises
a. live cell pellets (CP) of Lactococcus lactis subsp. lactis; and/or
b. fermented cell-free supernatant (CFS) comprising one or more metabolites produced by Lactococcus lactis subsp. lactis.

In an embodiments of the present disclosure, the composition comprises live CP of Lactococcus lactis subsp. lactis in an amount of 1%-99% w/v in a suitable solvent. For example, 1%, 2%, 3%, 4%, 5%, 6%, 7%, 8%, 9%, 10%, 11%, 12%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% w/v of the composition. In some embodiments, the composition comprises live CP of Lactococcus lactis subsp. lactis in an amount of 1%-50% w/v of the composition, 1%-20% w/v of the composition, 5-15% w/v of the composition, but not more than 20% w/v of the composition. In preferred embodiment, the composition comprises live CP of Lactococcus lactis subsp. lactis in an amount of 10% w/v of the composition.

In an embodiments of the present disclosure, the composition comprises CFS in a concentration of 1%-99% v/v in a suitable solvent. For example, 1%, 2%, 3%, 4%, 5%, 6%, 7%, 8%, 9%, 10%, 11%, 12%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% v/v of the composition. In some embodiments, the composition comprises CFS in a concentration of 1%-50% v/v of the composition, 1%-20% v/v of the composition, 5-15% v/v of the composition, but not more than 20% v/v of the composition. In preferred embodiment, the composition comprises CFS in a concentration of 10% v/v of the composition.

In an embodiment of the present disclosure, the solvent is a known solvent from prior arts. In some embodiments, the solvent is selected from but not limited to Tris-HCl Buffer, 0.85% saline, PBS, or water, or combinations thereof.

In a preferred embodiment, the present invention discloses the use of filtered CP and CFS that results in a complete (100%) reduction of FB1 compared to the initial mycotoxin concentration of 10 μg/ml (PBS buffer at pH 7.2).

In another preferred embodiment, a significant reduction of ZEA and DON was also observed, with CP treatment 72.30% reduction in ZEA and 31.58% reductions in DON were observed. Whereas with CFS treatment 40% reduction of ZEA, 50.78% of DON, and 37.63% reduction in OTA was noticed compared to the initial mycotoxin concentration.

In another embodiment, the present invention relates to the use of L. lactis subsp. lactis as a food and/or feed additive composition for the betterment of food safety. In some embodiments, the present invention relates to the use of live CP of Lactococcus lactis subsp. lactis; and/or fermented CFS of Lactococcus lactis subsp. lactis as a preservative for inhibition of microbial spore germination in a food product.

In an embodiments of the present disclosure, the food and/or feed additive composition comprises live CP of Lactococcus lactis subsp. lactis in an amount of 0.01%-0.5% w/w in a suitable solvent. For example, 0.01%, 0.02%, 0.03%, 0.04%, 0.05%, 0.06%, 0.07%, 0.08%, 0.09%, 0.1%, 0.11%, 0.12%, 0.13%, 0.14%, 0.15%, 0.16%, 0.17%, 0.18%, 0.19%, 0.20%, 0.3%, 0.4%, or 0.5% w/w of the composition. In preferred embodiment, the food and/or feed additive composition comprises live CP of Lactococcus lactis subsp. lactis in an amount of 0.125% w/w of the composition.

In an embodiments of the present disclosure, the food and/or feed additive composition comprises lyophilized CFS in an amount of 0.001%-0.010% w/w in a suitable solvent. For example, 0.001%, 0.002%, 0.003%, 0.004%, 0.005%, 0.006%, 0.007%, 0.008%, 0.009%, or 0.010% w/w of the composition. In preferred embodiment, the food and/or feed additive composition comprises lyophilized CFS in an amount of 0.005% w/w of the composition.

In an embodiment of the present disclosure, the solvent is a known solvent from prior arts. In some embodiments, the solvent is selected from but not limited to Tris-HCl Buffer, 0.85% saline, PBS, or water, or combinations thereof.

In another embodiment, the present invention relates to the process of detoxification of multiple mycotoxins such as FB1, ZEA, CIT, DON, and OTA comprising the steps of:
1. isolating bacteria from raw milk by homogenizing 9 ml of sterile distilled water containing 0.85% NaCl and 0.1% peptone, followed by 10-fold serial dilution from 10⁻¹ to 10⁻⁶ to form a culture;
2. fermentating, Lactococcus lactis subsp. lactis cultures in fermentation medium containing 100 ml De Man, Rogosa, and Sharpe (MRS) broth at 32 to 40° C. for 24 to 48 h;
3. centrifuging, at 4000 rpm for 20 min (Thermo Fisher Scientific, USA) to harvest CP and CFS;
4. collecting, the obtained supernatant in sterile fresh glass tubes and washing the biomass (cell pellet; CP) three times with PBS to remove any residual culture medium, and again centrifuging the residual culture to remove the water content;
5. detoxifying, mycotoxins using CP (0.5 to 0.1 g) and CFS (100 μl) by adding 1 mL PBS with a defined concentration of 10 μg/ml for FB1, ZEA, OTA, CIT, and 50 μg/ml for DON;
6. incubating, the samples of step 3 for 6 to 12 h at 37° C. under the shaking condition (80 rpm) and after the completing the incubation period, filtering each sample with PTFE syringe filters containing 0.22-μm-diameter pores; and
7. quantifying, the mycotoxins of the above samples by high-performance liquid chromatography (HPLC).

In some embodiments, the fermentation medium can also be selected from GM17 media, Elliker broth media and Whey-Based Media.

In an embodiment of the present invention, the strain Lactococcus lactis subsp. lactis as disclosed in the present invention is capable of producing novel metabolites such as 2,4-di-tert-butylphenol (1), 3,5-dihydroxy-6-methyl-2,3-dihydro-4H-pyran-4-one (2). Pyrazine-2-carboxamide (3).

In an embodiment the various metabolites produced from Lactococcus lactis subsp. lactis are present in CSF and are responsible for the detoxification of multiple mycotoxins of food product.

In an embodiment of the present invention, the food product is a natural food, food grains, processed food, ready-to-eat food, packaged food, stored food, and the like.

In an embodiment of the present invention, the food product is an animal feed product.

### EXAMPLES

The disclosure will now be illustrated with working examples, which are intended to illustrate the working of disclosure and not intended to take restrictively to imply any limitations on the scope of the present disclosure. Unless defined otherwise, all technical and scientific terms used herein have the same meaning as commonly understood to one of ordinary skill in the art to which this disclosure belongs. Although methods and materials similar or equivalent to those described herein can be used in the practice of the disclosed methods and compositions, the exemplary methods, devices, and materials are described herein.

### Experimental Methods

### 1. Sampling and Isolation of Bacteria

The traditional dairy milk samples were collected from Kolhapur, Maharashtra, India. The collected sample, 1 ml was homogenized with 9 ml of sterile distilled water containing 0.85% NaCl and 0.1% peptone. A 10-fold serial dilution was made from 10⁻¹ to 10⁻⁶ and, a 0.1 ml of appropriate dilutions was spread plated on Man, Ragosa, and Sharpe (MRS) agar plate and incubated at 37° C. for 48 h under aerobic conditions. At the end of the incubation period, the visible colonies that appeared on the MRS plate were transferred to the freshly prepared MRS agar plate and pure colonies were preserved at 4° C. for further studies.

2. Probiotic Characterization of Lactococcus lactis Subsp. lactis (MG917752).

In the present invention, probiotic characterization of L. lactis was carried out as per the Indian council of medical research (ICMR) guidelines.

Sequences in Lactococcus lactis subsp. lactis
BIONCL 17752 (16S rRNA sequence):
AACGTGGCAAGTTGAGCGCTGAAGGTTGGTACTTGTACCAACTGGATGAG
CAGCGAACGGGTGAGTAACGCGTGGGGAATCTGCCTTTGAGCGGGGGACA
ACATTTGGAAACGAATGCTAATACCGCATAAAAACTTTAAACACAAGTTT
TAAGTTTGAAAGATGCAATTGCATCACTCAAAGATGATCCCGCGTTGTAT
TAGCTAGTTGGTGAGGTAAAGGCTCACCAAGGCGATGATACATAGCCGAC
CTGAGAGGGTGATCGGCCACATTGGGACTGAGACACGGCCCAAACTCCTA
CGGGAGGCAGCAGTAGGGAATCTTCGGCAATGGACGAAAGTCTGACCGAG
CAACGCCGCGTGAGTGAAGAAGGTTTTCGGATCGTAAAACTCTGTTGGTA
GAGAAGAACGTTGGTGAGAGTGGAAAGCTCATCAAGTGACGGTAACTACC
CAGAAAGGGACGGCTAACTACGTGCCAGCAGCCGCGGTAATACGTAGGTC
CCGAGCGTTGTCCGGATTTATTGGGCGTAAAGCGAGCGCAGGTGGTTTAT
TAAGTCTGGTGTAAAAGGCAGTGGCTCAACCATTGTATGCATTGGAAACT
GGTAGACTTGAGTGCAGGAGAGGAGAGTGGAATTCCATGTGTAGCGGTGA
AATGCGTACATATATGGAGGAACACGGATGGCGAACGCGTCTCTCTGGCC
TGTATCTGACACTGAGGCTCGATAGCGTGGGGGAGCAAACGATATTAGAT
ACGGTGGTTCTCCACACCTCAACCGCATTAGAGCGGTGCAAAGGGAAGTC
CACTCTCTCTGTCTCGGAATTCAAGGCAAACAGTTTTCCAGGGGGGGCAG
GGGGAGAACCACTGGCATGTTCACACGAATTGAGAAATGCCCCGGGGCCC
CGCTTTACCCCCGAAAAATTCTGGGAAAACTGCGAGGAAACCTCGTAAAA
ACCTGGGGGGGTCGGGGAAGTAATTCGGGCCATTCCTTTGAGGGGGGGAT
TCCCCTTCGGGGACAGGGTTTCCCGTTTTGACAAGGTTTTTTTCCCCCCA
GGGGTGTTTAAGTTCCGAAAAACCTCCCCCCCCCCCGGGGGGCCTCTGTG
GGTAAATTTTTTTCTTTTTTGGGGAAAAAATCCCCAGGGGGGCCGCCAGA
AGGGGGGGGGGGGGGGGGCTCCCCCCCCGGGGGCCCCCCCCCCTCCGGGG
GGGAATTTGCCCGGGGGGGGGACACCCACCCAAAAAAAAAAAGAGTTTTT
TTTGAGTTTTTTGTTTTTTGAAAAACGGTTTATTTTTTTAGGCTCATTTT
TCACCCCCCCCACAAAAAGCCCCCCCCCCCCCCTTTGTGGGAGGAAAAAA
TCCCTCCGGGGGGGGGGGGGGGGTGGGGGCGATAAAAAAAAAAAAAAAAA
ATG

### 3. Molecular Identification of Bacteria

The isolated bacterium was identified by molecular identification by Polymerase Chain Reaction (PCR). The genomic DNA was extracted from the overnight grown culture using a genomic DNA extraction Kit (GenElute, Sigma Aldrich) as per the manufacturer's instructions and quantified using a Nano-drop spectrophotometer (Thermo Scientific, USA). The PCR run was performed in a 20 μL reaction with 16S rRNA universal primers 27 F 5′-AGAGTTTGATCM (A/C) TGGCTCAG-3′ and 1492 R 5′ TACCTTGTTACGACTT-3′ (Jiang et al. 2006).

### 4. Mycotoxin Detoxification

The Cell pellet (0.1 g) and cell-free supernatant (100 μl) was added to the 1 mL PBS with a defined concentration of 10 μg/ml for FB1, ZEA, OTA, CIT, and 50 μg/ml for DON. These samples were further incubated for 12 h at 37° C. under the shaking condition (80 rpm). After the completion of the incubation period, each sample was filtered with PTFE syringe filters containing 0.22-μm-diameter pores (Millex-GS, Millipore, USA). The two controls namely, a defined concentration of mycotoxins, and without mycotoxin in PBS used as positive and negative controls, respectively.

### 5. Mycotoxin Analysis by HPLC

Quantification of mycotoxins of the above samples was processed by high-performance liquid chromatography (HPLC) analysis. Waters 2545 Quaternary Gradient Module (Waters, USA) was used with an X-bridge C18 column (5 μm, 4.6×250 mm) for mycotoxin separation. The HPLC parameters used in the present study are presented in Table 1. The identification of mycotoxins was carried out by comparing the retention times of the peak of test samples with the standard mycotoxin solutions. To determine the mycotoxin concentration, the peak area of the test samples was correlated with the standard curves which were obtained by HPLC analysis of standard mycotoxin solutions. Each experiment was carried out in triplicate and results were obtained by taking arithmetic mean values.

TABLE 4
HPLC parameters used for mycotoxin analysis

| HPLC | Mycotoxins | | | | |
|---|---|---|---|---|---|
| parameters | FB1 | ZEA | DON | OTA | CIT |
| Mobile | 0.1 M | Acetonitrile/ | Acetonitrile/ | Acetonitrile/ | Acetonitrile/ |
| phase | Phosphate | water/ | water | water/acetic | water |
| | buffer/ | methanol | (10:90) | acid | (55:45) |
| | methanol | (46:46:08) | | (49:49:02) | |
| | (25:75) | | | | |
| Fluorescence | Excitation:335 | — | — | — | — |
| detector | Emission: 440 | | | | |
| λ (nm) | | | | | |
| UV | — | 236 | 218 | 333 | 254 |
| detector | | | | | |
| λ (nm) | | | | | |
| Flow | 1 | 0.5 | 0.6 | 0.5 | 0.7 |
| (ml/min) | | | | | |
| Injection | 20 | 20 | 20 | 20 | 20 |
| volume | | | | | |
| (μl) | | | | | |
| Run time | 15 | 15 | 10 | 15 | 15 |
| (minutes) | | | | | |

### 6. Fermentation Conditions

L. lactis culture was grown in de Man Rogosa Sharpe medium (MRS) using a 10 L fermenter (New Brunswick BioFlo/CelliGen115, Germany). The fermentation conditions were maintained as pH 6.5, incubation temperature 37° C., and 10% dissolved oxygen (DO) saturation with constant agitation at 75 rpm was optimized as ideal conditions. During the incubation period, the absorbance was measured every 2 h at 600 nm, when the absorbance was reached maximum (after 24 h) the fermentation process was stopped. After a successful fermentation batch, CFS from fermentation broth was harvested by centrifugation at 4000×g for 20 min.

7. Identification and Characterization of Bioactive Molecules from Lactococcus lactis Subsp. lactis

The CFS obtained from the L. lactis subsp. lactis was processed for extraction with ethyl acetate and dichloromethane (1:1). The obtained extract was concentrated using a rotary evaporator and analyzed by TLC. Based on the band observed in TLC, major bands were further purified by column chromatography and purified compounds were identified by nuclear magnetic resonance spectroscopy (NMR), X-ray diffraction (XRD), and liquid chromatography with a high-resolution mass spectrometer (LC-HRMS).

### Extraction:

2,4-di-tert-butylphenol (1): (0.230 g) as white solid. TLC: Rf=0.5 (SiO₂, 5% EtOAc/hexanes); 1H NMR (CDCl₃, 400 MHz): δ7.30 (d, J=2.4 Hz, 1H), 7.07 (dd, J=2.4, 8.3 Hz, 1H), 6.59 (d, J=8.3 Hz, 1H), 4.68 (s, 1H), 1.42 (s, 9H), 1.29 (s, 10H); ¹³C NMR (CDCl₃, 101 MHz): δ 151.9, 143.1, 135.3, 124.2, 123.7, 116.1, 34.9, 34.4, 31.8, 29.8. 3,5-dihydroxy-6-methyl-2,3-dihydro-4H-pyran-4-one (2): (0.145 g) as yellowish oil. TLC: Rf=0.5 (SiO₂, 50% EtOAc/hexanes); 1H NMR (CDCl₃, 400 MHz): δ4.54-4.34 (m, 2H), 4.03 (dd, J=10.2, 12.1 Hz, 1H), 2.10 (s, 3H); ¹³C NMR (CDCl₃, 101 MHz): δ 188.1, 160.3, 131.4, 71.1, 67.2, 15.9. HR-ESI m/z [M+H]⁺ (calcd for C₆H₉O₄, 145.0493; found 145.0495). Pyrazine-2-carboxamide (3): (0.035 g) as white solid. TLC: Rf=0.5 (SiO₂, 80% EtOAc/hexanes); ¹H NMR (CDCl₃, 400 MHz): δ9.42 (d, J=1.3 Hz, 1H), 8.78 (d, J=2.5 Hz, 1H), 8.58-8.53 (m, 1H), 7.65 (br. s., 1H), 5.91 (br. s., 1H); ¹³C NMR (CDCl₃, 101 MHz): δ165.5, 147.7, 144.8, 144.3, 142.9. HR-ESI m/z [M+H]⁺ (calcd for C₅H₆N₃O, 124.0505; found 124.0505).

### 8. Single-Crystal XRD Analysis of Bioactive Compound

a. The single crystal X-ray diffraction measurements were performed to determine the crystal structure of compounds 1, 2a and 3 at 100 K using APEX3 (Bruker, 2016; Bruker D8 VENTURE Kappa Duo PHOTON II CPAD) diffractometer having graphite-monochromatized (MoKα=0.71073 Å).
b. The X-ray generator was operated at 50 kV and 30 mA. A preliminary set of unit cell parameters and an orientation matrix were calculated from 36 frames, and the cell refinement was performed by SAINT-Plus (Bruker, 2016).
c. An optimized strategy used for data collection consisted of different sets of φ and scans with 0.5 steps φ/ω. The data were collected with a time frame of 10 sec for both the components by setting the sample to detector distance fixed at 40 cm.
d. All the data points were corrected for Lorentzian, polarization, and absorption effects using SAINT-Plus and SADABS programs (Bruker, 2016). SHELXS-97 (Sheldrick, 2008) was used for structure solution, and full-matrix least-squares refinement on F2.1, 2 The molecular graphics of ORTEP diagrams were performed by Mercury software.
e. The crystal symmetry of the components was cross-checked by running the cif files through PLATON (Spek, 2020) software and notified that no additional symmetry was observed. The Encifer software was used to correct the cif files.
   9. Antimicrobial Activity of Bioactive Molecules of Lactococcus lactis Subsp. lactis
a) The minimum inhibitory concentration (MICs) of purified compounds 1-3 were evaluated antimicrobial activity against Staphylococcus aureus NCIM 2079, Bacillus subtilis NCIM 2010, Escherichia coli NCIM 2065, Serratia marcescens NCIM 2919, Pseudomonas desmolyticum NCIM 2112, and Mycobacterium smegmatis NCIM 5138 whereas, antifungal activity was carried out against mycotoxigenic Fusarium verticillioides BIONCL4, Fusarium graminearum MTCC1893, Aspergillus niger BIONCL 12 and opportunistic pathogenic yeast, Candida albicans NCIM 3557 according to the clinical and laboratory standards institute (CLSI) guidelines (CLSI, 2008).
b) The stock solution was prepared by dissolving individual compounds in dimethyl sulfoxide (DMSO). The different concentrations of compound (0 to 100 μg mL⁻¹) and 50 μl bacterial suspension (5×10⁵ CFU mL⁻¹) were added to each well of a 96-well microtiter plate except for media control.
c) The broth, untreated culture, DMSO, and streptomycin sulphate were used as negative and positive controls. The final volume was adjusted to 200 μL with growth medium and incubated for 24 h at 37° C. under shaking conditions. At the end of the incubation, OD at 600 nm was measured using a multimode plate reader (Bio-Rad, USA).
d) The antimicrobial activity for M. smegmatis was carried out using the above protocol with Middlebrook 7H9 medium (Hi-Media, Mumbai, India), and incubated for 48 h.
e) Antifungal activity, RPMI-1640 (Sigma Aldrich, India) was used as a growth medium. The compounds with different concentrations as described above and 50 μl suspension of fungal spores (1×10⁶ mL-1) were added to each well of a 96-well microtiter plate except for media control, and incubated for 48 to 72 h at 28° C. under the shaking conditions. The media broth, untreated culture, DMSO as a control, and amphotericin B were used as standard drug.
f) Furthermore, following the above experimental plates, resazurin dye (Resazurin sodium salt, 2.5 mg mL-1) were added and incubated under dark conditions for 4 h. All the plates were protected by aluminium foil since resazurin is light-sensitive dye. The colour transition from pink to blue is considered to be MICs.
   10. Inhibitory Effect of Lactococcus lactis on FB1 Biosynthesis in F. verticillioides
a. Fusarium verticillioides growth inhibition was carried out using different concentrations of lyophilized CFS (0.1 to 0.5%) of L. lactis in 100 mL potato dextrose broth (PDB) and inoculated with fungal spores (107 spores/mL).
b. The flasks were incubated at 28° C. for 7 days. The flasks without CFS treatment serve as a control. At the end of the incubation, fungal mycelia was harvested and dried in a hot air oven at 50° C. for 2 h to remove the moisture content, and weighed for each treatment group and compared with control.
c. Fungal mycelia were crushed in liquid nitrogen using mortar and pestle was used for RNA extraction, followed by cDNA synthesis and FUM1 gene expression study using qRT-PCR.
   11. Effect of Lactococcus lactis on Growth and FB1 Production by Fusarium verticillioides in Stored Maize Grains
a. Samples of maize grains (500 g) were sterilized by autoclaving and divided into two groups: a treatment group was sprayed with 10 mL of lyophilized CFS (1.25 mg/g) and CP (50 μg/g) separately, and a control group was sprayed with 10 mL of sterile water and were dried in an incubator at 50° C. for 30 min.
b. Then, both groups were inoculated with a suspension containing 1 mL of F. verticillioides spores (10³ spore/g).
c. After 10 days of interval, a sample was taken and analyzed for spore germination using PDA agar plates and FB1 production using HPLC.

### Experimental Results

### 12. Neutralization of Mycotoxins

In this invention ion, in-vitro mycotoxin detoxification of various mycotoxins such as FB1, ZEA, CIT, DON, and OTA was processed.

After 12 h of incubation CP and CFS of L. lactis, resulted complete (100%) reduction of FB1, ZEA, and CIT.

Moreover, CP treatment resulted in a 31.58% reduction in DON, and an 18% reduction in OTA was observed.

Whereas, CFS treatment neutralized 50.78% of DON, and a 23% reduction in CIT was observed when compared to the control group (Table 2).

TABLE 5
In vitro detoxification of mycotoxins by
Lactococcus lactis subsp. lactis

| | Detoxification (%) | |
|---|---|---|
| Mycotoxins | CP | CFS |
| Fumonisin B1 | 100 | 100 |
| Zearalenone | 100 | 100 |
| Citrinin | 100 | 100 |
| Deoxynivalenol | 31.58 | 50.78 |
| Ochratoxin A | 18.00 | 23.00 |

A comparable table of earlier reports on L. lactis CP treatment results in the reduction of FB1 (100%), ZEA (30-88%), OTA (59%), and DON (11%) was reported (Table 6).
Whereas, there was no report found about CIT detoxification using LAB.
Compared to the previous reports, our present invention also showed the complete detoxification of FB1.
No reports were stating about the complete detoxification of ZEA and CIT.
Interestingly, we found a 100% reduction in ZEA and CIT and a 31-50% reduction in DON followed by an 18-23% reduction in OTA observed using L. lactis CP and CFS (Table 5).

TABLE 6
Comparative examples of mycotoxin detoxification using Lactococcus lactis
subsp. lactis with known literature methods

| | | | Mycotoxin | |
|---|---|---|---|---|
| | | Treatment | detoxification | |
| Name of the Bacteria | Mycotoxin | method | (%) | Reference |
| L. lactis | ZEA | Cell culture 2 ml | 88 | Krol et al., |
| | | (9.78 × 10⁸ CFU/ | | 2018 |
| | | mL) + 130 μg/mL | | |
| L. lactis | ZEA | Live/lyophilized | 47.4 | Rogowska |
| | | Cell pellet (18 × | | et al., 2019 |
| | | 10⁸ CFU/mL) 10 | | |
| | | ml sterile media + | | |
| | | ZEA (2 μg/mL) | | |
| L. lactis 56 KY484989 | ZEA | — | 30 | Zloch et al., |
| | | | | 2020 |
| L. lactis 202 | OTA | Live cell | 59.6 | Piotrowska |
| | | (10⁸ CFU/mL) | | et al., 2005 |
| | | culture media + | | |
| | | 10 μg/mL OTA | | |
| L. lactis | FB1 | Live cell in 2 mL | 100 | Niderkorn |
| | | MRS + FB1 (10 | | et al., 2006 |
| | | μg/mL) | | |
| Lactococcus | | Live cells in MRS | 100 | Niderkorn |
| lactis subsp. cremoris | FB1 | broth | | et al., 2009 |
| (cell wall components) | | (10¹⁰ CFU/mL)/ | | |
| | | cell wall | | |
| | | component + FB1 | | |
| | | (5 μg/mL) | | |
| Lactococcus genus | ZEA, DON, | | 17, 11, 18 | Niderkorn |
| | FB1 | | | et al., 2006 |
| L. plantarum | DON | Live cells | 28-35 | Zou et al., |
| | | | | 2012 |
| P. pentosaceus | DON | Live bacterial | 47 | Juodeikiene |
| | | suspension | | et al., 2018 |
| Lactococcus | ZEA | Live cells in PBS | 100 | Present |
| lactis subsp. lactis | FB1 | | 100 | Invention |
| (MG917752) | CIT | | 100 | |
| | DON | | 31-50 | |
| | OTA | | 18-23 | |

The comparative examples provided in the above table 6, indicates that the previous study stated about the mycotoxin detoxification potential of Lactococcus lactis subsp. lactis such as ZEA (17-88%), DON (11%), FB1 (100%) and OTA (59%) were reported. Whereas, present invention claims that Lactococcus lactis subsp. lactis strain having ability for the complete detoxification of ZEA and FB1 (100%), followed by DON (31-50%) and OTA (18-23%). Interestingly, there were no report on CIT detoxification using Lactococcus lactis subsp. lactis strains. Thus, present invention is a first report of covering the Lactococcus lactis subsp. lactis with have for the complete detoxification of CIT. Moreover, present study used live cells of Lactococcus lactis subsp. lactis in PBS buffer and there no further requirement of growth media and freeze-drying that indirectly minimize the process cost. In addition, strain used in the present invention showed probiotics characteristics and it is safe for humans and animals consumption.

### 13. Isolation, and Characterization, and Structure Elucidation of Bioactive Metabolites:

### 2,4-di-tert-butylphenol (1)

2,4-di-tert-butylphenol (1) was obtained as a white solid, and HRMS data determine the molecular formula C₁₄H₂₂O (m/z 207.17 [M+H]+) (calcd for C₁₄H₂₃O, 206.17) suggesting four indices of hydrogen deficiency. The structure of 1 was predominantly recognized on the basis of NMR spectroscopy, including ¹H and ¹³C NMR, COSY, HMQC, HMBC, and NOESY spectra. The 1H NMR displayed six methyl groups [δ_{H}1.42 (H-9), 1.29 (H-10)] and three aromatic protons [δ_{H}7.30 (H-3), 7.07 (H-5), 6.59 (H-6)]. The ¹³C NMR and HSQCNMR spectroscopic data exposed fourteen carbon, and found a six-methyl attached [δ_{c} 31.8 (C-9), 29.8 (C-10) attached with four quaternary carbon [δ_{c} (135.0) C-2, (143.1) C-4, 34.8 (C-7), 34.9 (C-8)] and one phenolic carbon [δ_{c} 151.9 (C-1)]. However, The HMBC correlation shows the connection between H-9 (δ_{H}1.42) to C-1 (δ_{C} 151.93), C-2 (δ_{c} 135.0), C-3 (δ_{c} 123.7), C-7 (δ_{c} 34.8) and H-10 (δ_{H}1.29) to C3 (δ_{c} 123.7), C-4 (δ_{c} 143.1), C-5 (δ_{c} 124.2), C-8 (δ_{c} 34.9) carbon shows a clear connection between the C1-C₂-C₃, C4-C5-C6. The Key ¹H-¹H COSY, NOESY, HSQC, and HMBC correlation established the position of di-tert-butyl group at C2 and C4 respective to the phenolic group at C1. A signal-crystal X-ray diffraction analyses of 2,4-di-tert-butylphenol was prepared from Hexane (100%), which led to the establishment of the complete structure.

### 3,5-dihydroxy-6-methyl-2,3-dihydro-4H-pyran-4-one (2)

3,5-dihydroxy-6-methyl-2,3-dihydro-4H-pyran-4-one (2) was obtained as a white solid, and HRMS data determine the molecular formula C₆H₈O (m/z C₆H₉O [M+H]⁺) Found 145.0495 (calcd for C₆H₉O, 145.0493) suggesting three indices of hydrogen deficiency. The ¹H NMR data showed signals of methylene proton [δ_{H}4.48 (H-2)], oxygen-bearing methine proton at [δ_{H}4.01 (H-3), and methyl proton at [δ_{H}2.10 (H-7)]. The ¹³C NMR and HSQC spectra of 2 revealed the presence of 6 carbons, including one carbonyl carbons [&c188.1 (C-4)], two olefin carbon [δ_{c} 160.3 (C-6), 131.4 (C-5), one Oxygen bearing methine carbon [δ_{c} 71.1 (C-3) one methylene carbon [δ_{c} 67.27 (C-2) and one methyl carbon [δ_{c} 15.9 (C-7). In the ¹H-¹H COSY spectrum, the correlations of an oxygen-bearing methine proton H-3 (δ_{H} 4.01) with methylene proton-2 (δ_{H} 4.48) indicated the linkage of C-2/C-3. In the HMBC spectrum, correlations of methylene proton H-2 (δ_{H} 4.5) at C-3 (δ_{c} 71.1), C-4 (188.1), C-6 (δ_{c} 160.3), oxygen-bearing methine proton-3 (δ_{H} 4.01) at C-2 (δ_{c} 67.9), C-4 (δ_{c} 188.1) methyl proton H-7 (δ_{H}2.10) at C-5 (δ_{c} 131.4) (δ_{c} 160.27) existing the presence of pyran ring. The structure of the 3,5-dihydroxy-2-methyl-4H-pyran-4-one resembles compound 2, except for the presence of a double bond at the 5th position of the hydroxyl group. As a result, the crystal structure of 3, 5-dihydroxy-2-methyl-4H-pyran-4-one gives insight into the metabolic activity of compound 2. The crystal structure of compound 2 could not be determined because crystals do not exist.

### Pyrazine-2-Carboxamide (3)

Pyrazine-2-carboxamide (3) was obtained as a white solid, and HRMS data determine the molecular formula C₅H₅N₃O (m/z C₅H₆N₃O [M+H]⁺) found 124.0505 (calcd for 124.0505) suggesting three indices of hydrogen deficiency. The ¹H NMR data showed the presence of aromatic proton at [δ_{H}9.42 (H-3), (8.56 (H-6), 8.77 (H-7)] and ¹³C NMR and HSQC spectra of compound 3 revealed the presence of 5 carbons, including one carbonyl carbons [δc 165.5 (C-7), three aromatic protons [δc 144.8 (C-2), 147.7 (C-5), 142.9 (C-6)] and one quaternary carbon at [δc 144.28 (C-2)]. In the ¹H-¹H COSY spectrum, the correlations of an aromatic proton H-5 (δ_{H} 8.77) with H-6 (δ_{H}8.56) indicated the linkage of C-5/C-6. In the HMBC spectrum, correlations of aromatic H-3 (δ_{H}9.42) proton at C-2 (δc 144.3), C-5, (δc 147.7), H-5 (δ_{H} (8.77) at C-2 (δc 144.3), C-3 (δc 144.8), C-6, (δc 142.9) and H-6 (δ_{H} 8.56) at C-2 (δc 144.3), C-6 (δc 142.9), existing the presence of pyrazine ring. A signal-crystal X-ray diffraction analyses of pyrazine-2-carboxamide was prepared from DCM:Hexane (10%), which led to the establishment of the complete structure.

TABLE 10
Crystallographic information details of compounds 1, 2 and 3.

| Crystal data | 1 | 2 | 3 |
|---|---|---|---|
| Chemical | C₁₄H₂₂O | C₆H₆O₄ | C₅H₅N₃O |
| formula | | | |
| Formula | | 142.11 | 123.12 |
| weight (Mr) | | | |
| Crystal system | Triclinic | Monoclinic | Monoclinic |
| Space group | P-1 | P2₁/n | P2₁/c |
| Temperature T | 100 | 100 | 100 |
| (K) | | | |
| a (A) | 13.174 (3) | 6.9540 (5) | 14.340 (2) |
| b (Å) | 13.306 (3) | 6.0448 (5) | 3.6313 (5) |
| c (A) | 13.489 (3) | 13.9726 (11) | 10.6302 (14) |
| α (°) | 111.026 (7) | 90 | 90 |
| β (°) | 104.030 (7) | 92.862 (3) | 101.184 (5) |
| γ (º) | 105.310 (7) | 90 | 90 |
| Z | | 4 | 4 |
| Volume (Å³) | 1973.8 (7) | 586.61 (8) | 543.05 (13) |
| Source of | MoKa | MoKa | MoKa |
| radiation | | | |
| Dcalc (Mg m⁻³) | 1.041 | 1.609 | 1.506 |
| Crystal size | 0.3 × 0.12 × | 0.16 × 0.1 × | 0.16 × 0.08 × |
| (mm) | 0.1 | 0.06 | 0.06 |
| μ (mm⁻¹) | 0.06 | 0.14 | 0.11 |
| Data | | | |
| collection | | | |
| Diffractometer | Bruker D8 | Bruker D8 | Bruker D8 |
| | VENTURE | VENTURE | VENTURE |
| | Kappa Duo | Kappa Duo | Kappa Duo |
| | PHOTON II | PHOTON II | PHOTON II |
| | CPAD | CPAD | CPAD |
| Absorption | Multi-scan | Multi-scan | Multi-scan |
| correction | (SADABS; | (SADABS; | (SADABS; |
| | Bruker, 2016) | Bruker, 2016) | Bruker, 2016) |
| Tₘᵢₙ, Tₘₐₓ | 0.6513, 0.7456 | 0.669, 0.746 | 0.606, 0.746 |
| No. of | 91696, 8515, | 27307, 1280, | 22725, 1183, |
| measured, | 5600 | 1240 | 1137 |
| independent | | | |
| and | | | |
| observed [ I > | | | |
| 2σ(I)] | | | |
| reflections | | | |
| Theta range | 2.50-27.33 | 2.92-30.19 | 2.90-27.50 |
| (°) | | | |
| Rᵢₙₜ | 0.191 | 0.060 | 0.071 |
| Refinement | | | |
| R[F² > | 0.163, 0.414 | 0.055, 0.108 | 0.036, 0.095 |
| (F²)], wR(F²) | | | |
| GOF on F² | 1.09 | 1.24 | 1.12 |
| No. of | 8515 | 1280 | 1183 |
| independent | | | |
| reflections | | | |
| No. of | 409 | 94 | 83 |
| parameters | | | |
| F_000 | 684 | 296 | 256 |
| No. of | 0 | 0 | 0 |
| restraints | | | |
| H-atom | Constr | Constr | Constr |
| treatment | | | |
| Δρₘₐₓ, Δρₘᵢₙ | 1.26, −1.35 | 0.34, −0.27 | 0.31, −0.25 |
| (eA°⁻³) | | | |
| CCDC number | | | |

TABLE 11
Hydrogen-bond geometry (A°, °) of compound 1, 2 and
3 are given as below.

| Name of the | | | | | | |
|---|---|---|---|---|---|---|
| compound | D—H . . . A | | D—H | H . . . A | D . . . A | D—H . . . A |
| 1 | C8—H8A | . . . O1 | 0.9800 | 2.3100 | 2.9227(7) | 120 |
| | C9—H9A | . . . O1 | 0.9800 | 2.3800 | 3.0134(7) | 122 |
| | C22—H22C | . . . O2 | 0.9800 | 2.3000 | 2.9672(7) | 124 |
| | C23—H23A | . . . O2 | 0.9800 | 2.3200 | 2.9618(7) | 122 |
| | C36—H36A | . . . O3 | 0.9800 | 2.3800 | 2.9846(7) | 119 |
| | C37—H37C | . . . O3 | 0.9800 | 2.3800 | 3.0147(7) | 122 |
| 2 | O2—H2 | . . . O3 | 0.8400 | 2.5200 | 2.8029(18) | 101 |
| | O2—H2 | . . . O3 | 0.8400 | 1.8500 | 2.658(2) | 161 |
| | O4—H4 | . . . O3 | 0.8400 | 2.4300 | 2.785(2) | 106 |
| | O4—H4 | . . . O3 | 0.8400 | 1.8900 | 2.694(2) | 160 |
| | C1—H1 | . . . O2 | 0.9500 | 2.3800 | 3.325(2) | 175 |
| | C6—H6B | . . . O4 | 0.9800 | 2.5200 | 2.898(3) | 103 |
| 3 | N2—H2A | . . . O1 | 0.8800 | 2.0300 | 2.9051(14) | 172 |
| | N2—H2B | . . . N1 | 0.8800 | 2.3700 | 2.7366(15) | 105 |
| | N2—H2B | . . . O1 | 0.8800 | 2.4600 | 3.1975(14) | 142 |
| | C3—H3 | . . . N1 | 0.9500 | 2.4700 | 3.3359(16) | 152 |
| | C4—H4 | . . . N3 | 0.9500 | 2.5900 | 3.4519(16) | 151 |
| | C5—H5 | . . . N3 | 0.9500 | 2.4500 | 3.3260(16) | 153 |

### 14. Antibacterial Activity

The isolated compounds were tested for antibacterial activity against both Gram-positive and Gram-negative bacteria. The compounds (1-3) have potent antibacterial activity against tested bacterial strains with MICs ranging from 1.56 to 25 μg/mL. All the investigated strains were significantly inhibited by compound C-1. The highest antibacterial activity was found against S. marcescens (MIC, 1.56 μg/mL), followed by M. smegmatis, E. coli, and B. subtilis (3.12 μg/mL), and least MIC was reported with S. aureus (6.25 μg/mL). The compound C-2 is active against S. marcescens, S. aureus with MIC value 3.12 μg/mL, and MICs for P. desmolyticum, E. coli, and B. subtilis (6.25 μg/mL), and least active against M. smegmatis (MIC, 12.5 μg/mL). Furthermore, compound C-3 is also more active against M. smegmatis with MIC (12.5 μg/mL), and other strains were less susceptible with MIC is 25 μg/mL was observed (Table 7).

### 15. Antifungal Activity

The compound C-1 is more active against F. verticillioides, F. graminearum showed highest inhibition with MIC of 6.25 μg/mL, A. niger, and C. albicans had shown least inhibition with MIC 12.5 μg/mL. The compound C-2 is active against all the fungal strains tested (MIC, 3.12 and 6.25 μg/mL), except F. graminearum. However, compound C-3 is exhibited good activity and MIC was found to be 12. 50 μg/mL against C. albicans, and F. verticillioides, other fungal stains tested were susceptible at 25.00 μg/mL was observed (Table 7).

TABLE 7
Antimicrobial activity of bioactive molecules isolated form
Lactococcus lactis

| | MIC(μg/mL) | | |
|---|---|---|---|
| | C-1 | C-2 | C-3 |
| Bacterial strain | | | |
| Staphylococcus aureus NCIM 2079 | 6.25 | 3.12 | 25.0 |
| Bacillus subtilis NCIM 2010 | 3.12 | 6.25 | 25.0 |
| Escherichia coli NCIM 2065 | 3.12 | 6.25 | 25.0 |
| Serratia marcescens NCIM 2919 | 1.56 | 3.12 | 25.0 |
| Pseudomonas desmolyticum NCIM 2112 | 6.25 | 6.25 | 25.0 |
| Mycobacterium smegmatis NCIM 5138 | 3.12 | 12.5 | 12.5 |
| Fungal strain | | | |
| Candida albicans NCIM 3557 | 12.5 | 3.12 | 12.5 |
| Fusarium verticillioides BIONCL4 | 6.25 | 3.12 | 12.5 |
| Fusarium graminearum MTCC1893 | 6.25 | 6.25 | 50.0 |
| Aspergillus niger BIONCL 12 | 12.50 | 3.12 | 25.0 |

C refers to the compound, MIC- Minimum inhibitory concentration, NCIM- National collection of industrial microorganism, MTCC- Microbial type culture collection.

16. Inhibitory Effect of Lactococcus lactis Subsp. Lactis on Fusarium verticillioides Growth and FB1 Biosynthesis

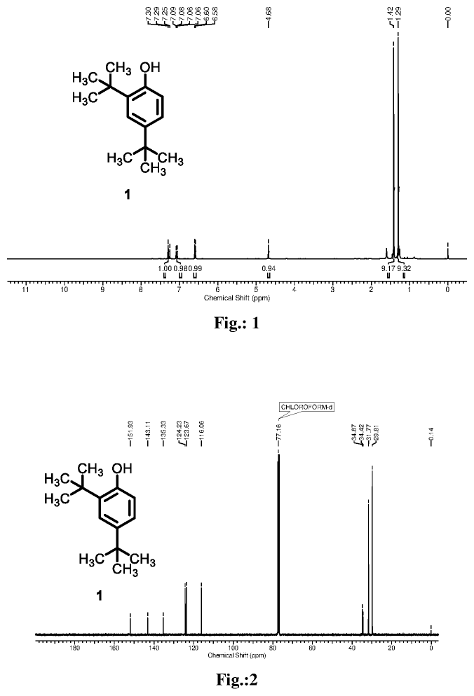

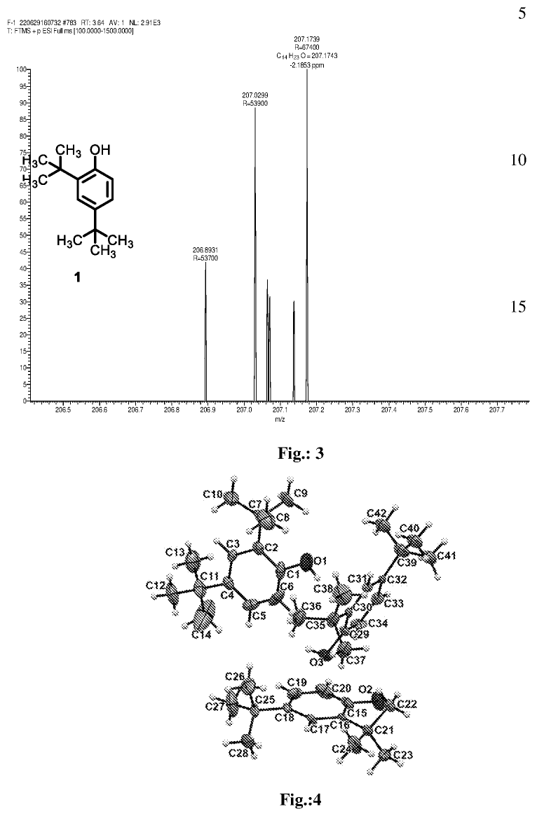

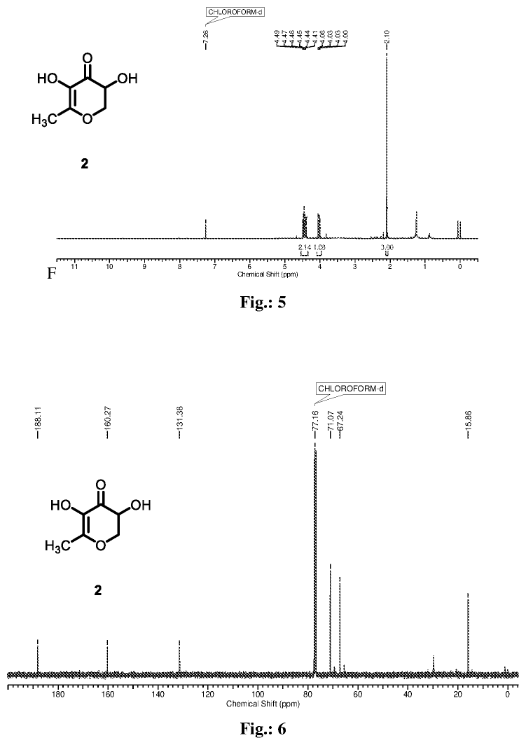

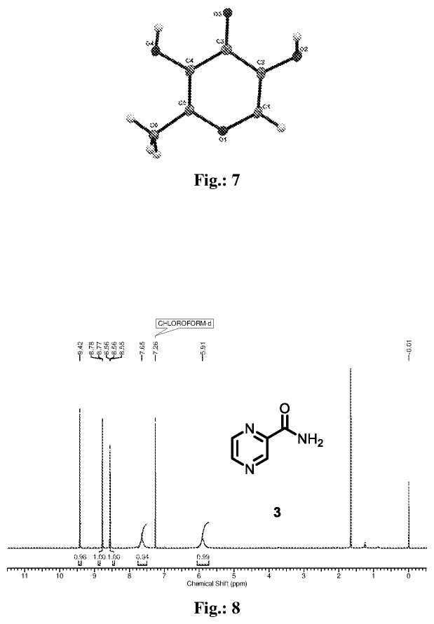

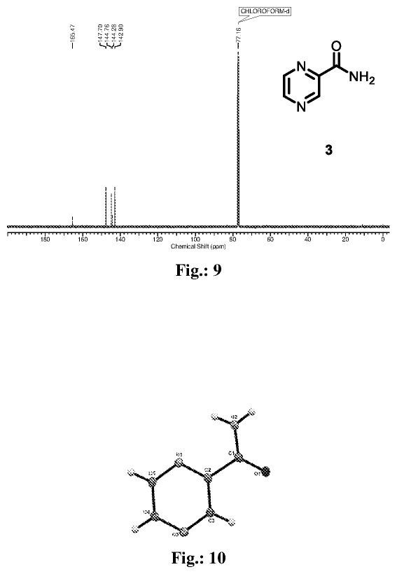

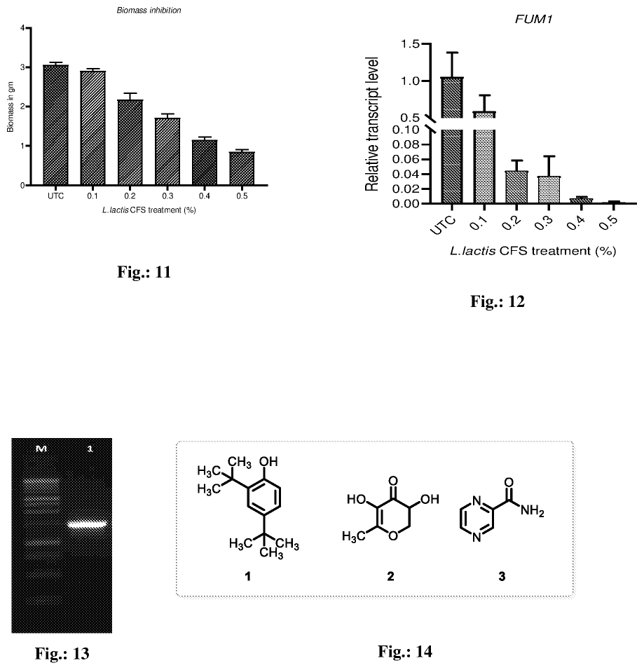

An in vitro inhibitory effect of L. lactis CFS on F. verticillioides growth and FUM biosynthesis showed that CFS treatment significantly inhibited the growth (72%) of F. verticillioides (FIG. 1). Interestingly, CFS treatment of L. lactis (0.1-0.5%) resulted in relative decrease in transcript level of FUM1 gene compared to untreated group demonstrated that CFS treatment significantly down regulated the FB1 gene expression in F. verticillioides (FIG. 1).

17. Effect of Lactococcus lactis Subsp. Lactis on Growth of F. verticillioides in Stored Maize Grains

The L. lactis CP and CFS treatment significantly inhibited about 43 to 64% spore germination of F. verticillioides and 100% FB1 production inhibition was observed in stored maize grain after 30 d of treatment (Table 8, 9). Moreover, it was found that at the end of 60 d, 40 to 61% spore germination and 74 to 85% FB1 production inhibition was recorded with CP and CFS treatment in maize grains (Table 8, 9). The obtained results stated that L. lactis CP and CFS could be used to improve the self-life of maize grains under storage condition.

TABLE 8
Effect of Lactococcus lactis subsp. lactis on spore
germination of Fusarium verticillioides

| | Inhibition (%) | |
|---|---|---|
| Time interval (days) | CP | CFS |
| 10 | 64.44 | 68.11 |
| 20 | 47.23 | 67.75 |
| 30 | 43.04 | 64.23 |
| 40 | 43.30 | 63.29 |
| 50 | 40.37 | 61.64 |
| 60 | 40.28 | 61.66 |

CP-cell pellet; CFS-cell free supernatant

TABLE 9
Effect of Lactococcus lactis subsp. lactis on
Fumonisin B1 (FB1) production by F. verticillioides

| | Inhibition (%) | |
|---|---|---|
| Time interval (days) | CP | CFS |
| 10 | 100 | 100 |
| 20 | 100 | 100 |
| 30 | 100 | 100 |
| 40 | 83.30 | 91.36 |
| 50 | 69.97 | 82.82 |
| 60 | 74.42 | 85.34 |

CP-cell pellet; CFS-cell free supernatant

### Advantages

Effectively provide a method for in-vitro detoxification of multiple mycotoxins using Lactococcus lactis subsp. lactis.
Environment friendly and economical method for effectively removing mycotoxin contamination and their risk to health/environment.
Effective neutralization of multiple mycotoxins in food and feed is crucial and the same is achieved by the present process.

1-14. (canceled)

15. A composition comprising:
(i) live cell pellets (CP) of Lactococcus lactis subsp. lactis having GenBank accession number MG917752; and
(ii) fermented cell-free supernatant (CFS) comprising one or more metabolites produced by Lactococcus lactis subsp. lactis in a solvent,
wherein the CP of Lactococcus lactis subsp. lactis is in a concentration in the range of 5-15% w/v; and the fermented cell-free supernatant (CFS) is in a concentration in the range of 5-15% v/v.

16. The composition of claim 15, wherein the solvent is selected from the group consisting of Tris-HCl Buffer, 0.85% saline, PBS, water, and combinations thereof.

17. The composition of claim 15, wherein the one or more metabolites produced by Lactococcus lactis subsp. lactis are selected from the group consisting of 3,5-dihydroxy-6-methyl-2,3-dihydro-4H-pyran-4-one, pyrazine-2-carboxamide, and 2,4-di-tert-butylphenol.

18. A process for production of the composition of claim 15, said process comprising the steps of:
(a) isolating Lactococcus lactis subsp. lactis (MG917752) from raw milk by serial dilution;
(b) fermenting Lactococcus lactis subsp. lactis (MG917752) of step (a) at a temperature of 32-37° C. for 24 to 48 hours in a growth medium;
(c) incubating Lactococcus lactis subsp. Lactis (MG917752) of step (b) followed by fermentation to obtain grown biomass crude mixture of Lactococcus lactis subsp. lactis based live cell pellets, and fermented cell-free supernatant (CFS) comprising one or more metabolites produced by Lactococcus lactis subsp. Lactis;
(d) filtering fermented broth of step (c) to separate live cell pellet (CP) containing Lactococcus lactis subsp. lactis (MG917752) and cell-free supernatant (CFS) of Lactococcus lactis subsp. lactis (MG917752); and
(e) preparing the composition by physical mixing of said live cell pellets (CP) containing Lactococcus lactis subsp. lactis (MG917752) and said cell-free supernatant (CFS) of Lactococcus lactis subsp. lactis (MG917752) of step (d) in a solvent to obtain the composition.

19. The process as claimed in claim 18, wherein the growth medium is selected from the group consisting of de Man Rogosa Sharpe medium (MRS), GM17 media, Elliker broth media, and Whey-Based Media.

20. The process as claimed in claim 18, wherein the physical mixing of step (e) comprises physical mixing of CP in a concentration of 5-15% w/v and fermented cell-free supernatant (CFS) in a concentration of 5-15% v/v, under stirring.

21. The process as claimed in claim 18, wherein said solvent for cell-free supernatant (CFS) of Lactococcus lactis subsp. lactis (MG917752) is phosphate-buffered saline (PBS) or water.

22. The process as claimed in claim 18, wherein the isolation of Lactococcus lactis subsp. lactis (MG917752) from raw milk comprises the steps of:
(a) providing a raw milk;
(b) homogenising the raw milk of step (a) with sterile distilled water containing 0.85% NaCl and 0.1% peptone;
(c) preparing serially diluted samples from the homogenized raw milk of step (b) and spreading the diluted samples on Man, Ragosa, and Sharpe (MRS) agar plate;
(d) incubating the MRS agar plates of step (c) at 32° C. to 37° C. for 24 to 48 h under aerobic conditions;
(e) picking up a typical Lactococcus characteristics colonies and stricked on MRS agar plates followed by Gram's staining and microscopic observation; and
(f) confirming the strains of L. lactis subsp. lactis (MG917752) by molecular identification using 16s RNA gene.

23. A process for in vitro detoxification of mycotoxin in a food product, said process comprising treating the composition of claim 17 with the food sample containing mycotoxins by spraying the composition onto said sample followed by incubating at a temperature in the range of 35 to 55° C. for a time period in the range of 0.5 to 24 hrs to obtain a detoxified product.

24. The process of claim 23, wherein the mycotoxins are selected from the group consisting of ochratoxin A (OTA), zearalenone (ZEA), fumonisins B1 (FB1), deoxynivalenol (DON), citrinin (CIT), and combinations thereof.

25. The process of claim 23, wherein the food product is selected from the group consisting of a natural food, food grains, processed food, ready-to-eat food, packaged food, and stored food.

26. A food additive comprising the composition of claim 15, which, when added to a food product, inhibits microbial spore germination in the food product.

27. The food additive of claim 26, wherein live cell pellets (CP) of Lactococcus lactis subsp. lactis are present in an amount of 0.01% to 0.5% w/w, and the cell-free supernatant (CFS) is present in an amount of 0.001% to 0.010% w/w, wherein the CFS is lyophilized CFS.

28. A preservative comprising the composition of claim 15.
